(19) United States
(12) Patent Application Publication
(10) Pub. No.: US 2013/0085485 A1
(43) Pub. Date: Apr. 4, 2013

(54) Systems and Methods for Treating Pathological Skin Conditions Using a Laser Diode

(71) Applicant: BIOLASE, Inc., Irvine, CA (US)

(72) Inventors: Marcia Van Valen, Aliso Viejo, CA (US); William E. Brown, JR., Roswell, GA (US)

(73) Assignee: BIOLASE, Inc., Irvine, CA (US)

(21) Appl. No.: 13/633,390

(22) Filed: Oct. 2, 2012

Related U.S. Application Data
(60) Provisional application No. 61/542,718, filed on Oct. 3, 2011.
(60) Provisional application No. 61/601,481, filed on Feb. 21, 2012.
(60) Provisional application No. 61/614,448, filed on Mar. 22, 2012.

(51) Int. Cl. A61B 18/20; A61B 18/22

(52) U.S. Cl. 606/9

(57) ABSTRACT

In accordance with the teachings described herein, systems and methods are provided for treating pathological skin conditions. A method of removing fungus from a treatment area may include placing an energy delivery apparatus on the treatment area, and activating the medical laser to deliver electromagnetic energy over an exposure distance to the treatment area in order to cause a temperature of tissue in the treatment area to rise to within a fungal treatment temperature range. The energy delivery apparatus may be attachable to an energy emitting portion of a medical laser and may be configured to position the energy emitting portion at the exposure distance from a surface of the treatment area when the energy delivery apparatus is placed on the treatment area.

### CROSS-REFERENCE TO RELATED APPLICATION

This application claims priority to the following three U.S. Provisional Patent Applications, each of which is incorporated herein by reference in its entirety: U.S. Provisional Patent Application No. 61/542,718, filed on Oct. 3, 2011; U.S. Provisional Patent Application No. 61/601,481, filed on Feb. 21, 2012; and U.S. Provisional Patent Application No. 61/614,448, filed on Mar. 22, 2012.

### FIELD

The technology described in this patent document relates generally to medical lasers, and more particularly, to the use of medical lasers in the treatment of pathological skin conditions.

### BACKGROUND

Pathological skin conditions, such as fungal diseases, are not uncommon in humans. Fungi tends to thrive in environments that are dark, warm, and moist, such as those environments found under and around toenails. While topical medication can be used to treat such fungal conditions, topical medication is typically required to be applied twice daily for an extended period of time, such as one year. The discipline necessary to conform to this regimen often leads to lapses in patient compliance with an attendant lack of success in treatment. Oral antifungal medications such as terbinafine or intraconazole are sometimes used but may have side effects such as recurrent infection and are typically not recommended in patients with certain medical conditions or those taking certain medications. Surgery is another option but severe fungus may require complete removal of the nail and may take a year or more to grow back.

A need exists in the context of treating pathological skin conditions, such as topical or persistent fungal conditions, for an efficient and effective non-medicinal or only partially medicinal method of treating such diseases of the skin. A commensurate need exists for a medical device capable of implementing such improved non- or partially-medical methods.

### SUMMARY

In accordance with the teachings described herein, systems and methods are provided for treating pathological skin conditions. A method of removing fungus from a treatment area may include placing an energy delivery apparatus on the treatment area, and activating the medical laser to deliver electromagnetic energy over an exposure distance to the treatment area in order to cause a temperature of tissue in the treatment area to rise to within a fungal treatment temperature range. The energy delivery apparatus may be attachable to an energy emitting portion of a medical laser and may be configured to position the energy emitting portion at the exposure distance from a surface of the treatment area when the energy delivery apparatus is placed on the treatment area.

A system for removing fungus from a treatment area may include a medical laser and an energy delivery apparatus. The medical laser may be configured to emit electromagnetic energy from an energy emitting portion. The energy delivery apparatus may be detachably coupled to the energy emitting portion of the medical laser and configured to position the energy emitting portion at an exposure distance from a surface of the treatment area when the energy delivery apparatus is placed on the treatment area. The medical laser may be configured to focus electromagnetic energy in a uniform beam from the energy emitting portion onto a surface of the treatment area such that a laser spot size of the uniform beam on the surface of the treatment area is dependent on the exposure distance.

A disposable energy delivery apparatus may include a proximal portion, a distal portion and a body portion. The proximal portion may be configured to detachably couple the disposable energy delivery apparatus to an energy emitting portion of a medical laser. The distal portion may be configured to be positioned against a surface of a fungal treatment area. The body portion may connect the proximal portion to the distal portion such that the disposable energy delivery apparatus, when coupled to the energy emitting portion of the medical laser, is configured to position the energy emitting portion at an exposure distance from a surface of the treatment area when the energy delivery apparatus is placed on the treatment area.

### BRIEF DESCRIPTION OF THE DRAWINGS

FIG. 1 is a block diagram of an example system for treating a pathological skin condition, such as a fungal infection.

FIGS. 2 and 3 are block diagrams of more particular examples of systems for treating a pathological skin condition, such as a fungal infection.

FIG. 4 is a flow diagram of an example method for laser nail fungus treatment.

FIG. 5 is a diagram of an example laser handpiece with a detachable energy delivery apparatus.

FIG. 6 depicts another example energy delivery apparatus attached to a laser handpiece.

FIGS. 7A and 7B depict another example of an energy delivery apparatus.

FIG. 8A and 8B include a cross-sectional depiction of an example energy delivery apparatus and laser handpiece.

### DETAILED DESCRIPTION

FIG. 1 is a block diagram of an example system 100 for treating a pathological skin condition, such as a fungal infection. The system 100 includes an electromagnetic energy generator 110 and an energy delivery apparatus 120 for delivering electromagnetic energy to a treatment area 130 over a fixed exposure distance 140. The electromagnetic energy generator 110 may be a medical laser which may, for example, generate electromagnetic energy using a laser diode. According to a particular implementation, the electromagnetic energy generator 110 may be a medical laser that includes a laser diode that generates electromagnetic energy having a wavelength that ranges from about 405 nm to about 1320 nm. The power level of the electromagnetic energy generated by the electromagnetic energy generator 110 may range from about 0.3 W to about 40 W in continuous or pulsed mode when measured at a predetermined exposure distance 140 (e.g., 30 mm) from the surface of the treatment area 130.

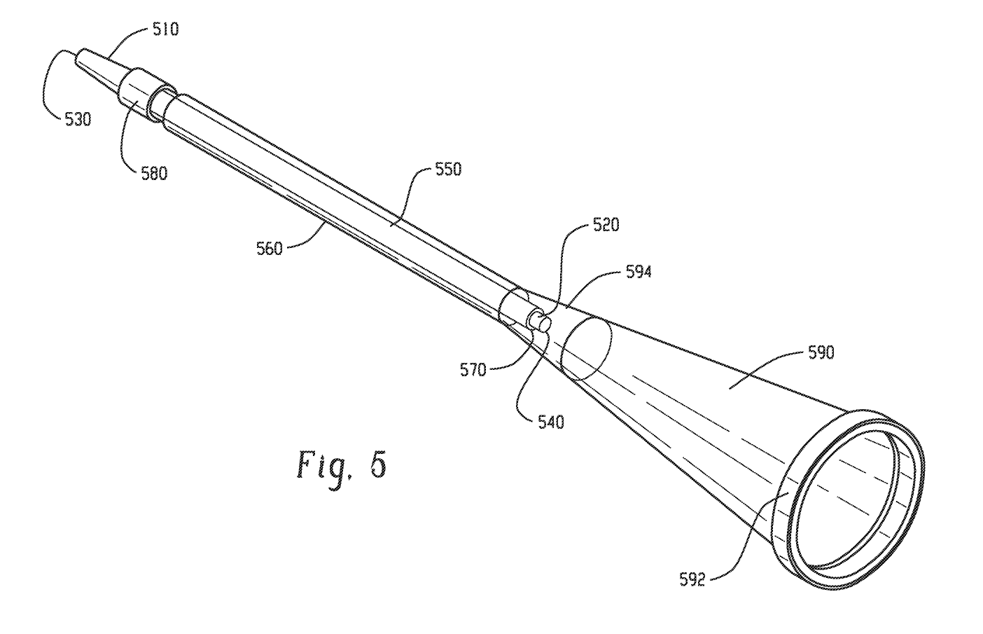

The energy delivery apparatus 120 is configured to deliver the electromagnetic energy over the predetermined exposure distance 140 to the treatment area 130. The electromagnetic energy may be focused into a uniform beam having a predetermined spot size at the exposure distance 140, for example using an optical lens in either the electromagnetic energy generator 110 or the energy delivery apparatus 120. The energy delivery apparatus 120 may, for example, be a disposable adapter (e.g., as shown in FIGS. 5-8) that attaches to the handpiece of a medical laser and that is configured to be placed onto the surface of the treatment area 130 in order to maintain a predetermined exposure distance 140 between the treatment area 130 and an energy emitting optic lens. For instance, the medical laser and energy delivery apparatus 120 may be configured to deliver a uniform beam with a 30 mm spot size to the treatment area over a 30 mm exposure distance. In this manner, a consistent level of electromagnetic energy may be deployed to the treatment area 130 in order to cause the temperature of tissue within the treatment area 130 to be maintained within a fungal treatment temperature range. A temperature range of about 40°-48° C. (104°-118.4° F.), and preferably about 40°-40.5° C. (104°-105° F.), will typically kill fungus in the treatment area without causing thermal damage to living tissue. In addition, fungus treatment may also result from a fungicidal effect from the specific laser wavelength as well as biostimulation of the surrounding tissue.

In one example, the system 100 may utilize a 940 nm diode laser, such as the Diolase 10™ laser sold by Biolase, Inc. of Irvine, Calif. A 940 nm laser can be employed to generate an electromagnetic energy beam which may be particularly well-suited for the treatment of a fungal infection because of its absorption by hemoglobin, oxyhemoglobin, melanin, and its fungicidal effect. Fungus, being a systemic disease, affects tissue and underlying tissue where hemoglobin, oxyhemoglobin, melanin, and its fungicidal effect exist. The reaction of a 940 nm laser with the hemoglobin and oxyhemoglobin in the treatment area may thus be utilized to elevate the temperature of the underlying blood supply to within the desired fungal treatment range. For instance, a 40.5° C. fungal treatment temperature may be reached by directing a 940 nm laser (e.g., a Diolase 10™ laser) with a spot size of about 30 mm on the treatment area for about 30 seconds. Firing the laser at about 7.5 W for about 30 seconds may kill fungus when treatment is performed two times with a minimum of about 30 seconds between treatments. The 30 second waiting period allows thermal relaxation so that the temperature of the tissue does not rise above about 42° C., a temperature which may be considered to be within the “safe” zone for living tissue.

In one example, a 40° C. temperature was reached in a fungal treatment area in 25 seconds using a Diolase 10™ laser set on Type III skin (Fitzpatrick scale.) The procedure was repeated after 30 seconds. Set on Type I skin (Fitzpatrick scale), the Diolase 10™ laser reached the 40° C. temperature in 27 seconds and did not exceed this temperature. The procedure was repeated after 30 seconds. Before (i.e., initial) and after (i.e., three months) photographs of toenails treated using this protocol confirm the efficacy of the method during a one-year clinical testing period.

In a further example, toenail fungus was grown in a jar for 28 days and then lased with a Diolase 10™ laser at a setting of 7.5 W for 30 seconds. Lab testing showed that the fungus was alive prior to lasing and that the fungus was killed by lasing in a continuous mode for a period of 30 seconds on, 30 seconds off, and 30 seconds on again, or equivalent parameters in a pulsed mode.

In other examples, different laser wavelengths, such as wavelengths in the mid-infrared (e.g., around 2700 nm to around 3000 nm), such as Er,Cr:YSGG at 2,780 nm, may also be used at higher energy levels (e.g., around 0.25 Watts to around 9 Watts) to ablate and remove fungus. In another example, fungus may be ablated and removed using a solid state laser, such as a Nd:YAG or Nd:YAP laser with wavelengths of approximately 1,060 nm.

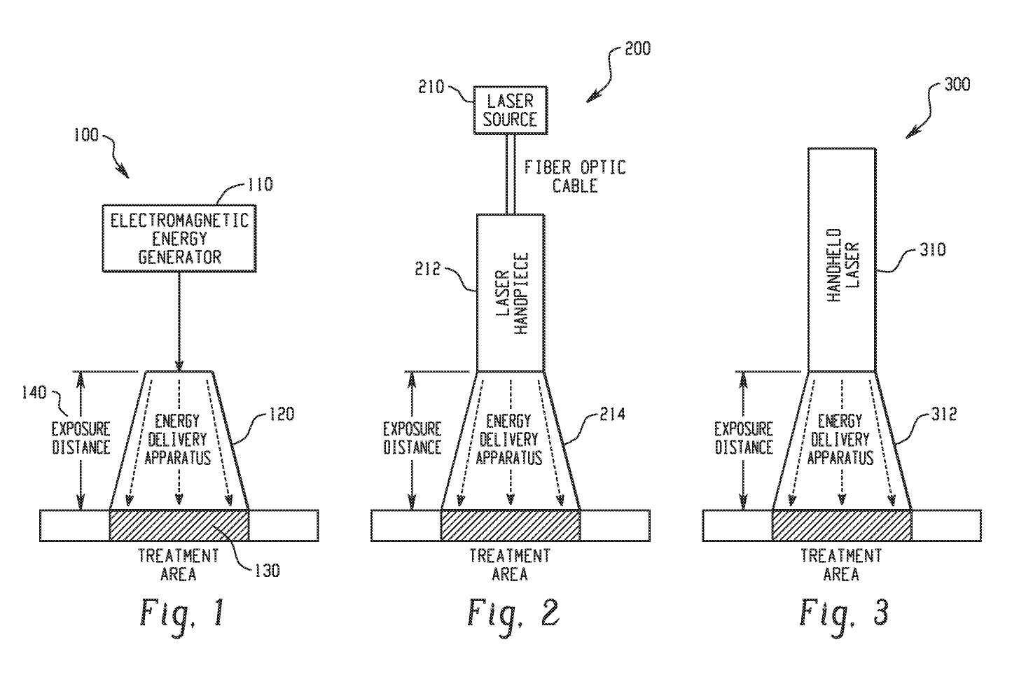

FIGS. 2 and 3 are block diagrams of more particular examples of systems for treating a pathological skin condition, such as a fungal infection. In the example illustrated in FIG. 2, the system 200 includes a medical laser source 210 having a detachable handpiece 212, and an energy delivery apparatus 214 that is detachably connected to the laser handpiece 212. In this example 200, the laser source 210 may include a laser housing having a laser therein that is capable of generating electromagnetic energy. The detachable handpiece 212 may be connected to the laser housing with a fiber optic cable that is configured to transmit electromagnetic energy from the laser source 210 to the laser handpiece 212. In the example illustrated in FIG. 3, the system 300 includes a handheld laser that is detachably connected to an energy delivery apparatus 312. In this example 300, a laser diode may be built into the handheld laser.

In both examples 200 and 300, the system is configured to transmit electromagnetic radiation from a laser in a uniform beam over an exposure distance from a proximal end to a distal end of the attached energy delivery apparatus 214, 312 in order to generate a radiation spot size (e.g., 30 mm spot size) on the surface of the treatment area. In certain preferred examples, the energy delivery apparatus 214, 312 is disposable (e.g., to prevent cross-contamination between patients.) The energy delivery apparatus 214, 312 may be manufactured of a material having a color or opaqueness to allow for the filtration of laser light. For instance, the energy delivery apparatus 214, 312 may be formed of an opaque, semi-transparent material (e.g., a material that is partially transmissive to visible light or to the laser wavelength being used.) In one example, the material of an outer wall of the energy delivery apparatus 214, 312 is a milky white semi-translucent structure.

In certain examples, the energy delivery apparatus 214, 312 may also be configured to contain particulates or gases (e.g., lasing byproducts) that are generated from the lasing of the fungal tissue. The energy delivery apparatus 214, 312 may include a plastic structure that is enclosed other than an opening at the distal end that is placed over the treatment area. Different embodiments of the energy delivery apparatus 214, 312 may include walls of a color and transmissivity that are selected so as not to inhibit the ability of the energy delivery apparatus 214, 312 to contain harmful particulates and gasses.

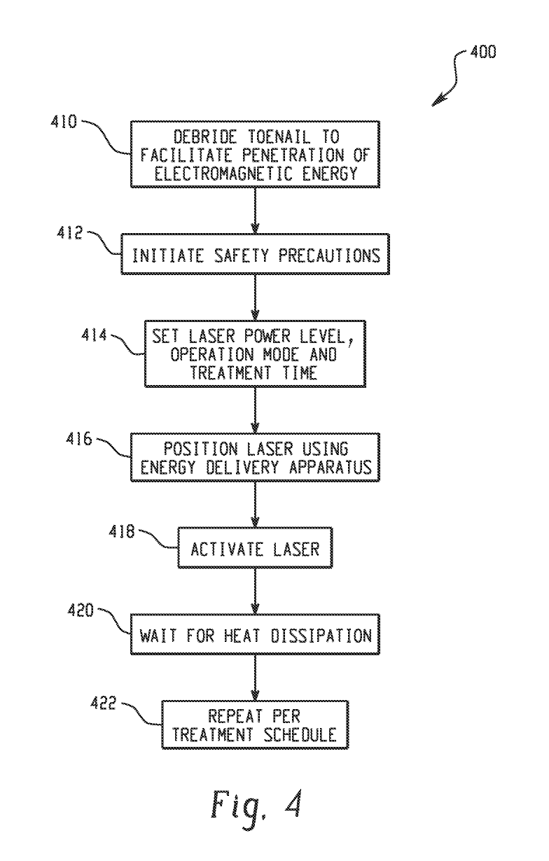

FIG. 4 is a flow diagram of an example method 400 for laser nail fungus treatment. At step 410, a fungus infected nail (i.e., toenail or fingernail) is debrided for treatment so as to augment efficacy. Debriding the nail for treatment may, for example, enable treatment energy to better penetrate the nail. In certain examples, a reactive dye may also be added to the treatment area. For example, a 940 nm wavelength laser will react with a purple ginseng die commonly used to mark surgical procedures. The dye may, for example, be applied to the treatment area in a mist or cotton swab prior to lasing.

Prudent safety precautions are initiated at 412. For example, assuming that a 940 nm laser is to be employed, appropriate eye protection should be placed on anyone within a safety perimeter (e.g., about 30 feet), a door to the treatment room should be closed, and a safety notification (e.g., a laser safety sign) should be put in place. At 414, the laser power level is set (e.g., to about 7.5 W), a laser mode is selected (e.g., continuous or pulsed mode), and a treatment time is selected (e.g., about 30 seconds.) In some examples, the laser may be operated in a pulsed mode, however, operation in a continuous mode is preferred.

At step 416, the laser is positioned for treatment using an energy delivery apparatus that maintains a consistent laser spot size on the treatment region. For instance, in one example the energy delivery apparatus is used to position the laser perpendicular to the nail at a predetermined exposure distance. The exposure distance may be selected to cause the electromagnetic energy to be emitted in a uniform beam having a spot size at the distal end of the energy delivery apparatus that covers the treatment area (e.g., a 30 mm laser spot size that covers the entire toenail and nailbed.) Examples of an energy delivery apparatus are described below with reference to FIGS. 5-8.

At step 418, the laser is activated by, for example, depressing a foot pedal and waiting until a countdown reaches zero. Because laser energy applied to a nail is expected to increase the temperature of the nail, step 420 prescribes waiting until built-up heat is dissipated (e.g., about 30 seconds in one implementation.) The treatment may then be repeated at step 422. In one example, the lasing treatment (e.g., steps 418 and 420) may be repeated a plurality of times to maintain a desired treatment temperature within the treatment area. For example, a treatment regimen may include activating the laser for 30 seconds, followed by 30 seconds off to allow heat to dissipate, followed by another 30 seconds of lasing.

In addition, because of the characteristics of fungus, which is a systemic disease, the fungus may tend to return if conditions that allowed it to manifest are not changed. Accordingly, for this or other reasons, multiple treatments may be necessary and ongoing. The treatment method 400 may therefore be repeated over a treatment schedule. For instance, a first year treatment schedule may include a total of six treatments per treatment session, repeated at one week, one month, three months, and then continuing every three months thereafter for management of a fungus condition. More severe fungus may require more initial treatments before the three month management treatment schedule is enacted. Toenails, for example, can tend to grow relatively slowly (e.g., approximately 1 mm per month); thus conditions that allow the fungus to manifest may remain over time even with treatment, noting that the nail develops in the nailbed. Because there is approximately 3 mm of nail under the nailbed, a period of every three months for multiple treatments may be indicated or recommended.

FIG. 5 is a diagram of an example laser handpiece 560 with a detachable energy delivery apparatus 590. In the illustrated example, a portion 510 of an optical fiber 520 is disposed to receive electromagnetic energy at a first (e.g., proximal) end 530 of the handpiece 560 (i.e., the fiber comes through a top of the handpiece) from a laser source (not shown) and to convey the energy to a second (e.g., distal) end 540 of the handpiece 560. The fiber 520 may be contained in a casing 550, the casing 550 and fiber 520 being disposed inside the handpiece 560 positioned and supported by, e.g., plastic strips (not shown). The casing 550 may terminate at a distal end 570 through which may protrude a distal end of the fiber 520.

The laser handpiece 560 may further include ridges formed on an outside thereof in order to facilitate gripping of the handpiece 560. An attachment mechanism 580 disposed on the proximal end of the handpiece 560 may comprise an edge or a quick release button (not shown) to allow for quick disconnect of the handpiece 560 from the laser source when the button is pushed. A second quick release button may enable releasing of the energy delivery apparatus 590 thereby enhancing the disposability of the energy delivery apparatus 590.

Electromagnetic energy may be delivered from the optical fiber 520 to tissue through the energy delivery apparatus 590 (i.e., a delivery area where light is delivered), which may comprise a nominally atmospheric-containing (e.g., encircling) structure that couples to the handpiece 560 by a sheath 594. One implementation comprises the energy delivery apparatus 590 being constructed of a translucent or nominally cone-shaped structure that connects to the handpiece 560 by a clear sheath 594 to, inter alia, surround or protect part or all of the fiber when the handpiece 560 is attached. Example implementations may include a lasing-byproduct (e.g., particulate/gas) capturing and containing (e.g., cone-shaped) structure (e.g., a non-translucent tapering or conical structure) that connects to the handpiece 560 by a sheath 594.

During use, the delivery system (i.e., the delivery area inside the energy delivery apparatus 590) may light up. The sheath 594 may be relatively thick in order to provide insulation from heat (e.g., for a user's hand holding the system) or from lasing byproducts (e.g., particulates/gasses generated during lasing) while handling. The energy delivery apparatus 590 may terminate at a distal end in an open ring to which is disposed a foam pad or padded ring 592 that may be applied directly to (e.g., to contact) tissue being treated. The padded ring 592 may enhance treatment precision or patient comfort and discourage re-use of the energy delivery apparatus 590. The energy delivery apparatus 590, by way of including the padded ring 592, encloses the treatment area and provides the mentioned protection from breathing lasing byproducts (e.g., isolates) produced during treatment. That is, the full enclosure provides a level of protection for fungal particles in airways. When the energy delivery apparatus 590 is made of a translucent material, the property of helping to illuminate the area being treated may be enhanced, whereby facilitating visualization of the treatment area can provide guidance to a doctor and to a patient. Embodiments of the energy delivery apparatus 590 may be formed of semi-translucent material of various visually-distinguishable colors, too, in order to enhance viewing of a treatment area.

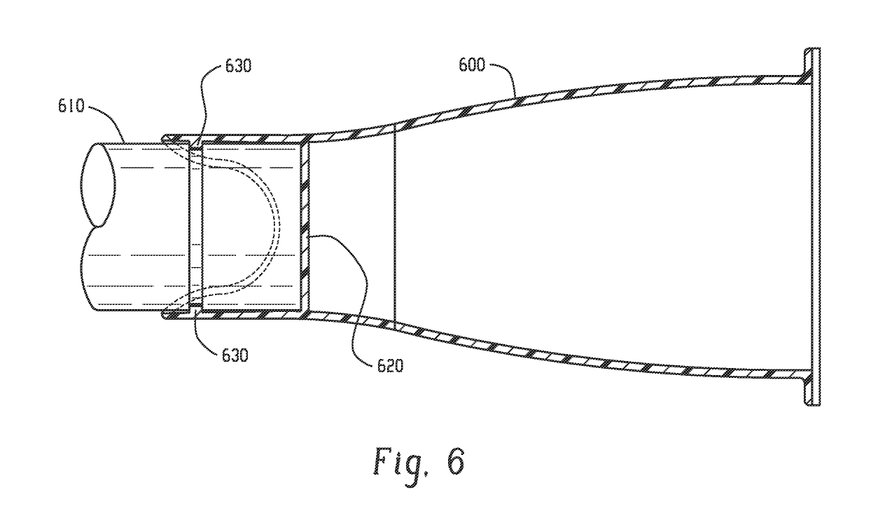

FIG. 6 depicts another example energy delivery apparatus 600 attached to a laser handpiece 610. In this example, the energy delivery apparatus 600 includes a protective barrier 620 at the proximal end for preventing lasing byproducts or other materials from contacting the lens or fiber optic cable in the laser handpiece 610. Also included in this example are detent clips 630 on the energy delivery apparatus 600 that are received in a slot within the laser handpiece 610 in order to easily attach the energy delivery apparatus 600 to the laser handpiece 610.

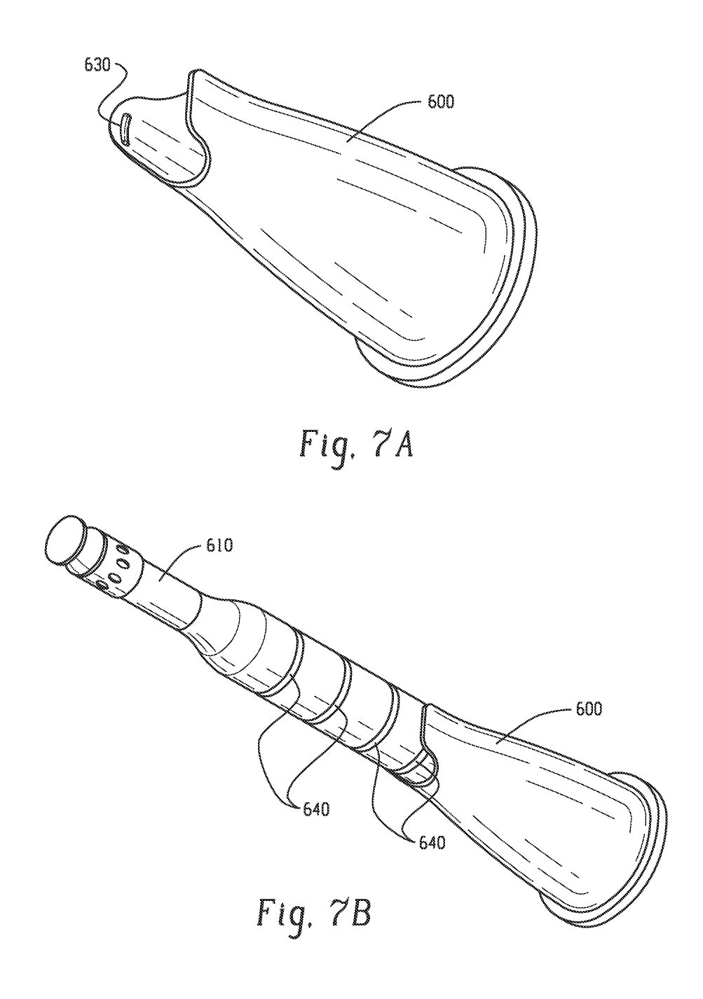

Another example of the example energy delivery apparatus 600 is illustrated in FIGS. 7A and 7B. FIG. 7A shows an example of the energy delivery apparatus 600 by itself, and FIG. 7B shows another example of the energy delivery apparatus 600 attached to a laser handpiece 610. FIG. 7A illustrates an example of the detent clips 630 that are used to attach the energy delivery apparatus 600 to a laser handpiece. In the example illustrated in FIG. 7B, the laser handpiece 610 includes multiple slots 640 for receiving the detent clips 630 on the energy delivery apparatus 600. In this manner, the slots 640 in the laser handpiece 610 may be used to select a desired exposure distance between the energy-emitting end of the laser handpiece 610 and the treatment area. For instance, in the illustrated example, the energy delivery apparatus 600 is attached to a 30 mm slot in the laser handpiece 610, but could also be attached to slots for selecting a 15 mm, 20 mm or 25 mm exposure distance.

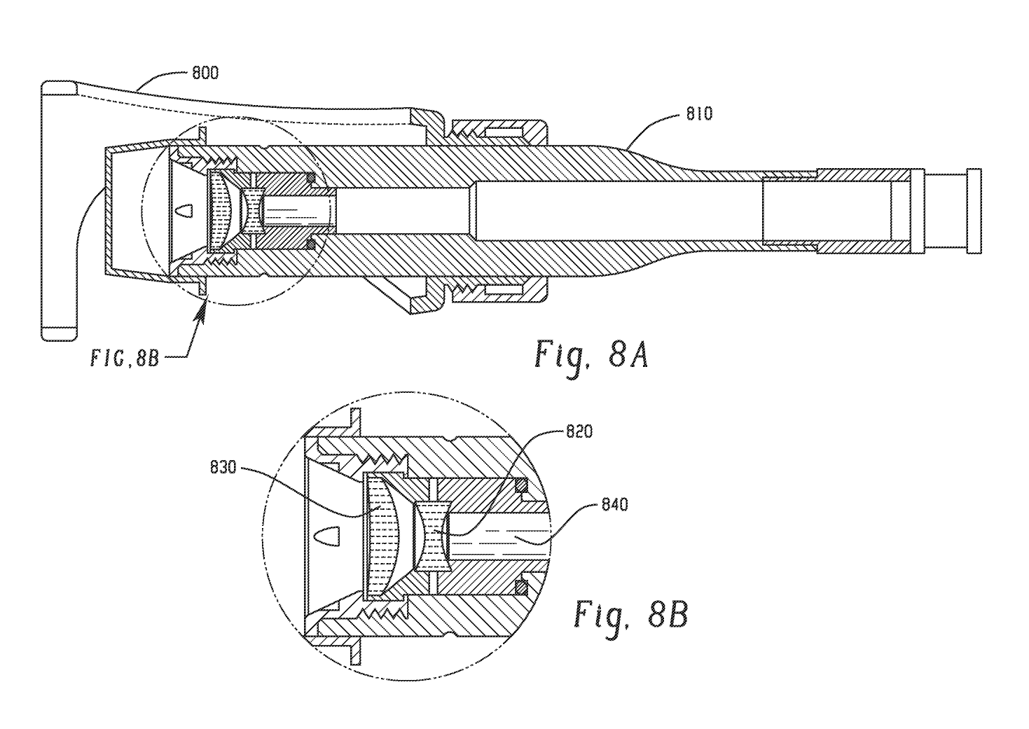

FIG. 8A is a cross-sectional depiction of an example energy delivery apparatus 800 and laser handpiece 810. FIG. 8B provides a magnified view of a light emitting end of the laser handpiece 810. As shown in FIG. 8B, the laser handpiece 810 may include one or more lens 820, 830 for focusing the emitted electromagnetic energy into a desired beam width in order to provide a desired laser spot size over a fixed exposure distance. In the illustrated example, the laser handpiece 810 includes an optic fiber 840 that directs electromagnetic energy from a laser source to a biconcave lens 820, and a meniscus lens 830 that refocuses electromagnetic energy from the biconcave lens 820 to achieve a desired spot size.

While the disclosure has been described in detail and with reference to specific embodiments thereof, it will be apparent to one skilled in the art that various changes and modifications can be made therein without departing from the spirit and scope of the embodiments. Thus, it is intended that the present disclosure cover the modifications and variations of this disclosure provided they come within the scope of the appended claims and their equivalents.

It should be understood that as used in the description herein and throughout the claims that follow, the meaning of “a,” “an,” and “the” includes plural reference unless the context clearly dictates otherwise. Also, as used in the description herein and throughout the claims that follow, the meaning of “in” includes “in” and “on” unless the context clearly dictates otherwise. Further, as used in the description herein and throughout the claims that follow, the meaning of “each” does not require “each and every” unless the context clearly dictates otherwise. Finally, as used in the description herein and throughout the claims that follow, the meanings of “and” and “or” include both the conjunctive and disjunctive and may be used interchangeably unless the context expressly dictates otherwise.

It is claimed:

1. A method of removing fungus from a treatment area, the method comprising:
placing an energy delivery apparatus on the treatment area, the energy delivery apparatus being attachable to an energy emitting portion of a medical laser and being configured to position the energy emitting portion at an exposure distance from a surface of the treatment area when the energy delivery apparatus is placed on the treatment area; and
activating the medical laser to deliver electromagnetic energy over the exposure distance to the treatment area, the electromagnetic energy causing a temperature of tissue in the treatment area to rise to within a fungal treatment temperature range.

2. The method of claim 1, wherein the medical laser is configured to focus electromagnetic energy in a uniform beam from the energy emitting portion onto the surface of the treatment area, and wherein a laser spot size of the uniform beam on the surface of the treatment area is dependent on the exposure distance.

3. The method of claim 1, wherein the energy delivery apparatus is configured to enclose the treatment area such that gaseous or airborne byproducts generated by exposure of the tissue to the electromagnetic energy are captured and contained by the energy delivery apparatus.

4. The method of claim 1, further comprising:
deactivating the medical laser after a first time duration;
after deactivating the medical laser, waiting a second time duration without the medical laser activated; and
reactivating the medical laser, and leaving the laser activate for a third time duration.

5. The method of claim 4, wherein the first, second and third time durations are selected to prevent the temperature of the living tissue from rising above the fungal treatment temperature range.

6. The method of claim 1, wherein the electromagnetic energy has a wavelength ranging from about 405 nm to about 1320 nm.

7. The method of claim 1, wherein the electromagnetic energy has a wavelength ranging from about 2700 nm to about 3000 nm.

8. The method of claim 1, wherein the electromagnetic energy has a power level ranging from about 0.3 Watts to about 40 Watts.

9. The method of claim 1, wherein the electromagnetic energy has a power level ranging from about 0.25 Watts to about 9 Watts.

10. The method of claim 1, wherein the fungal treatment temperature ranging from about 40 degrees Celsius to about 48 degrees Celsius.

11. The method of claim 1, wherein the medical laser includes a laser diode for generating the electromagnetic energy.

12. The method of claim 1, wherein the medical laser includes one of a Nd:YAG, ND:YAP, Er, Cr:YSGG and Er:YAG laser.

13. The method of claim 1, wherein the energy emitting portion of the medical laser is a laser handpiece.

14. A system for removing fungus from a treatment area, the system comprising:
a medical laser configured to emit electromagnetic energy from an energy emitting portion; and
an energy delivery apparatus detachably coupled to the energy emitting portion of the medical laser and configured to position the energy emitting portion at an exposure distance from a surface of the treatment area when the energy delivery apparatus is placed on the treatment area,
wherein the medical laser is configured to focus electromagnetic energy in a uniform beam from the energy emitting portion onto a surface of the treatment area, and wherein a laser spot size of the uniform beam on the surface of the treatment area is dependent on the exposure distance.

15. The system of claim 14, wherein the medical laser comprises:
a laser housing that includes a laser that generates the electromagnetic energy; and
a laser handpiece that comprises the energy emitting portion, the laser handpiece coupled to the laser housing by an optic fiber that transmits the electromagnetic energy from the laser to the energy emitting portion.

16. The system of claim 15, wherein the energy emitting portion includes one or more lens for focusing the electromagnetic energy into the uniform beam.

17. The system of claim 14, wherein the medical laser includes a laser diode that generates the electromagnetic energy.

18. The system of claim 17, wherein the laser diode is configured to emit electromagnetic energy having a wavelength in a range of about 405 nm to about 1320 nm.

19. The system of claim 14, wherein the energy delivery apparatus is configured to enclose the treatment area such that gaseous or airborne byproducts generated by exposure of tissue to the electromagnetic energy are captured and contained by the energy delivery apparatus.

20. The system of claim 14, wherein an attachment between the energy delivery apparatus and the energy emitting portion of the medical laser is adjustable to select the exposure distance.

21. A disposable energy delivery apparatus system for attachment to a medical laser for removing fungus from a treatment area, the disposable energy delivery apparatus comprising:
a proximal portion configured to detachably couple the disposable energy delivery apparatus to an energy emitting portion of the medical laser;
a distal portion configured to be positioned against a surface of a the treatment area; and
a body portion connecting the proximal portion to the distal portion,
wherein the disposable energy delivery apparatus, when coupled to the energy emitting portion of the medical laser, is configured to position the energy emitting portion at an exposure distance from a surface of the treatment area when the energy delivery apparatus is placed on the treatment area.

22. The apparatus of claim 21, wherein the body portion of the energy delivery apparatus is configured to enclose the treatment area such that gaseous or airborne byproducts generated by exposure of tissue to the electromagnetic energy are captured and contained by the energy delivery apparatus.
